(19) United States
(12) Patent Application Publication
(10) Pub. No.: US 2023/0414719 A1
(43) Pub. Date: Dec. 28, 2023

(54) METHODS OF USING PLATELET FACTOR 4 AS AN ANTIMICROBIAL AGENT

(71) Applicant: Arizona Board of Regents on Behalf of Arizona State University, Scottsdale, AZ (US)

(72) Inventors: Tatiana UGAROVA, Phoenix, AZ (US); Nataly PODOLNIKOVA, Mesa, AZ (US)

(21) Appl. No.: 18/252,171

(22) PCT Filed: Nov. 9, 2021
(86) PCT No.: PCT/US21/58654

§ 371 Date: May 8, 2023

Related U.S. Application Data
(60) Provisional application No. 63/111,320, filed on Nov. 9, 2020.

(51) Int. Cl. A61K 38/19; A61K 45/06; A61P 31/04

(52) U.S. Cl. CPC A61K 38/195; A61P 31/04; A61K 45/06

(57) ABSTRACT

The present disclosure relates to methods of inhibiting and/or reducing the severity of a bacterial infection comprising the administration of Platelet Factor 4 (PF4). The bacterial infection can be caused by Gram-negative or Gram-positive bacteria. The bacterial infection can also have progressed to sepsis or peritonitis. In particular implementations, a method of inhibiting and/or reducing the severity of a Staphylococcus aureus infection is described, including a MRSA infection.

### RELATED APPLICATIONS

This application claims the benefit of and priority to U.S. provisional patent application 63/111,320, filed Nov. 9, 2020, the entirety of the disclosure of which is hereby incorporated by this reference thereto.

### STATEMENT REGARDING FEDERALLY SPONSORED RESEARCH OR DEVELOPMENT

This invention was made with government support under R01 HL063199 awarded by the National Institutes of Health. The government has certain rights in the invention.

### FIELD OF THE INVENTION

The invention relates to the use of Platelet Factor 4 (PF4) as an antimicrobial agent derived from PF4.

### BACKGROUND OF THE INVENTION

When Sir Alexander Fleming discovered penicillin, the first modern day antibiotics, in 1928, bacterial infections became less deadly ailments. Now, bacterial infections have again become possibly untreatable due to the rise of antibiotic resistance. Since 1941, when the “golden era” of antibiotics began with the mass-production of penicillin, more and more strains of bacteria became antibiotic-resistant and some have become resistant to multiple antibiotics resulting in the phenomenon of multidrug resistance (MDR). Widespread use of antibiotics has been implicated in the emergence of antibiotic-resistant bacteria that present a substantial threat to the successful treatment of infectious diseases. However, the prevailing natural phenomenon of survival means that bacteria will ultimately develop resistance to modern day antibiotics even with responsible antibiotic use.

Antibiotic resistance is a global issue affecting both human and animal health. According to the United States' Center for Disease Control and Prevention, at least 2.8 million people a year are infected with antibiotic-resistant bacteria or fungi in the United States, and among them, more than 35,000 people die. Globally, 700,000 deaths are due to antimicrobial resistance.

An especially serious threat is the emergence of Gram-negative bacteria that are resistant to essentially all of the available antimicrobial agents. A recent World Health Organization (WHO) report points that although multidrug-resistant Gram-positive bacteria remain on a high priority, research and development strategies should also focus on new agents that are specifically active against multidrug- and extensively drug-resistant Gram-negative bacteria. The WHO considers multidrug- and extensively drug-resistant Gram-negative bacteria to be critical priority in the search for new antimicrobial agents.

If the issue of antibiotic resistance is not addressed, many advances in modern medicine that depend on fighting infections may be jeopardized. In particular, without timely treatment with antibiotics, sepsis, the body's extreme response to an infection, can rapidly lead to tissue damage, organ failure, and death. Furthermore, patients who have surgery, organ transplant recipients, or people receiving chemotherapy for cancer are at risk for developing an infection and all depend on the treatment with antibiotics. Accordingly, there is a great need to discover and develop new antimicrobial agents.

### SUMMARY OF THE INVENTION

The disclosure relates to methods of inhibiting or treating a bacterial infection in a subject using Platelet Factor 4 (PF4, UniProtKB-P02776). In some aspects, the methods of reducing the severity of bacterial infection are disclosed. Also disclosed are methods of inducing an immune response in response to a bacterial infection, for example enhancing phagocytosis in response to a bacterial infection. The methods comprise administering to the subject PF4. In some aspects, the subject is administered recombinant PF4. The administration of PF4 reduces inflammation in the subject, for example, as manifested by a reduction in activated leukocytes or reduction in the level of circulating inflammatory cytokines. In some implementations, the method further comprises administering to the subject an antibiotic compound, a non-antibiotic antibacterial substance, and/or a glucocorticoid.

The disclosed methods inhibit bacterial infections caused by Gram-negative bacteria and by Gram-positive bacteria. In certain implementations, the bacterial infection is caused by Staphylococcus aureus, including methicillin-resistant S. aureus (MRSA). In some aspects, the subject has developed sepsis or peritonitis from the bacterial infection. In some embodiments, the bacterial infection is caused by an antibiotic-resistant bacteria. The antibiotic-resistant bacteria is resistant to at least one antibiotic selected from the group consisting of: penicillin, oxacillin, methicillin, and amoxicillin-clavulanic.

### BRIEF DESCRIPTION OF THE DRAWINGS

FIGS. 1A and 1B depict, in accordance with certain embodiments, the colony-forming units (CFU) recovered from lavage of mice inoculated with S. aureus alone (1×10⁷ colony-forming units, CFU) or with a combination of endotoxin-free recombinant PF4 (20 μg) and S. aureus (1×10⁷ CFU). FIG. 1A shows two exemplary agar plates, while FIG. 1B is a graph summary of the results.

FIG. 2 depicts, in accordance with certain embodiments, a comparison of leukocyte migration to the peritoneum 24 hours after intraperitoneal injection of control (PBS), PF4, and S. aureus alone (1×10⁷ colony-forming units, CFU) or with a combination of endotoxin-free recombinant PF4 (20 μg) and S. aureus (1×10⁷ CFU).

FIG. 3 depicts, in accordance with certain embodiments, the effect of PF4 on S. aureus in mouse at 24 hours after infection.

FIG. 4 depicts, in accordance with certain embodiments, an analysis of survival rate in mice inoculated with sublethal dose of dose of S. aureus (5×10⁸ CFU) with or without endotoxin-free PF4 (13 μg/mouse). The presence of PF4 decreased mortality from a S. aureus infection.

FIG. 5 depicts, in accordance with certain embodiments, a graph comparing the colony-forming units (CFU) recovered from lavage of mice inoculated with methicillin-resistant S. aureus (MRSA) alone (1×10⁷ colony-forming units, CFU) or with a combination of endotoxin-free recombinant PF4 (13 μg) and MRSA (1×10⁷ CFU).

### DETAILED DESCRIPTION OF THE INVENTION

Detailed aspects and applications of the invention are described below in the drawings and detailed description of the invention. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the invention. It will be understood, however, by those skilled in the relevant arts, that the present invention may be practiced without these specific details. It should be noted that there are many different and alternative configurations, devices, and technologies to which the disclosed inventions may be applied. The full scope of the inventions is not limited to the examples that are described below.

The singular forms “a,” “an,” and “the” include plural referents unless the context clearly dictates otherwise. Thus, for example, reference to “a step” includes reference to one or more of such steps.

As used herein, the term “antibiotic” or “antibiotic compound” refers to a naturally produce antibacterial substance. For example, the term includes, penicillin, tetracycline, erythromycin, methicillin, gentamicin, vancomycin, imipenem, ceftazidime, ceftriaxone, ampicillin, levofloxacin, linezolid, daptomycin, amphotericin B, cephalosporin, ceftaroline, and azithromycin.

As used herein, the term “non-antibiotic antibacterial substance” includes synthetic antibacterial substances, for example, sulfonamides or antiseptics.

As used herein, the term “subject” refers to a multi-cellular vertebrate organism, including human and non-human mammals, for example, mice.

As used herein, the term “inhibiting a bacterial infection” refers to reducing the quality, amount, or strength of viable bacteria in an infected subject. In one example, the inhibition of a bacterial infection includes decreasing or reducing one or more biological activities, such as growth, reproduction, proliferation, survival rate, metabolism, vitality, robustness, action, and/or function of microorganisms by at least 10%, at least 20%, at least 50%, or even at least 90%, including between 10% to 95%, 20% to 80%, 30% to 70%, 40% to 50%, such as 10%, 20%, 30%, 40%, 50%, 60%, 70%, 80%, 85%, 90%, 95%, 98%, or 100%. Such decreases can be measured using the methods disclosed herein as well as those known to one of ordinary skill in the art.

As used herein, the term “reducing the severity of a bacterial infection” refers to reducing the quality, amount, or strength of viable bacteria in an infected subject and to reducing the mortality rate of a bacterial infection.

Described herein is a novel antimicrobial therapeutic strategy based on Platelet Factor 4 (PF4, UniProtKB-P02776). The global challenges presented by drug-resistant bacteria stimulated the search for new direct-acting traditional antibiotics but also non-traditional options. The non-traditional options include targeting bacterial virulence, impeding bacterial adhesion to host cells and biofilm formation, interrupting or inhibiting bacterial communication, microbiome-modifying therapies, the employment of phages as treatments or carriers, and some immunomodulating strategies. Enhancing the activity of macrophages, professional phagocytes that play a crucial role in the host defense against microbial infection by ingesting and killing bacteria, has been a previously unexplored strategy.

The discovery that PF4 alone possesses antimicrobial properties arose from a strategy of targeting the outer capsule of bacteria that protects bacteria from engulfment by macrophages. This outer capsule is present in many Gram-negative and Gram-positive bacteria. The capsule is considered a virulence factor, because it allows bacteria to evade phagocytosis. The capsule comprises acidic polysaccharides, and the phagocytic receptor Mac-1 tends to not bind to negatively charged molecules.

PF4 is one of the most abundant cationic proteins secreted from α-granules of activated platelets. Based on its structure, PF4 was assigned to the CXC family of chemokines and has been shown to have numerous effects on myeloid leukocytes, including chemotaxis. PF4 induces leukocyte responses through the integrin Mac-1 (αMβ2, CD11b/CD18). Human neutrophils, monocytes, U937 monocytic and model HEK293 cells expressing Mac-1 strongly adhered to immobilized PF4 in a Mac-1-dependent manner. PF4 also induced the Mac-1-dependent migration of human neutrophils and monocyte/macrophages. Importantly, coating bacteria (for example, Escherichia coli and Staphylococcus aureus) or latex beads with PF4 enhanced their phagocytosis by macrophages by approximately 4 folds. This process was blocked by different Mac-1 antagonists. Furthermore, PF4 potentiated phagocytosis by wild-type, but not Mac-1-deficient macrophages. Thus, PF4 is a ligand for the integrin Mac-1, which suggests that many immune-modulating effects previously ascribed to PF4 were mediated through its interaction with Mac-1. PF4 directly binds to the αMI-domain, the major ligand-binding region, of Mac-1. Binding sites for αMI-domain were identified in two positively charged PF4 segments: Cys12-Ser26 and Ala57-Ser70. The presence of two binding sites allows for one of the sites to bind the negatively charged bacterial surface while the other side binds the macrophage receptor Mac-1. Thus, PF4 is an ideal opsonin that bridges bacteria to macrophages.

It was surprisingly discovered that PF4 alone can inhibit a bacterial infection or reduce the severity of the bacterial infection, and its mechanism of action is independent of targeting a specific species or class of bacteria. PF4 binds to the negatively charged surface of bacteria, and it is also a Mac-1 ligand. Thus PF4's interaction with the bacterial surface creates the binding sites for Mac-1 on macrophages and causes bacteria to lose their protective shield for evading phagocytosis. As shown in the examples, the presence of PF4 when there is a bacterial infection augments a subject's own defense against the infection, for example, the function of macrophages, in particular phagocytosis. PF4 dramatically augments bacterial phagocytosis by macrophages and reduces bacterial burden by 7-fold. Leukocyte migration in response to bacterial infection, particularly in a sepsis model, is also reduced in the presence of PF4.

Accordingly, described herein are methods of inhibiting a bacterial infection or methods of reducing the severity of a bacterial infection comprising administering to a subject in need thereof an antimicrobial agent derived from PF4. The bacterial infection may be caused by Gram-positive or Gram-negative bacteria. In some aspects, the bacterial infection is caused by S. aureus. Surprisingly, the administration of PF4 reduces the level of inflammation caused by the bacterial infection. As shown in the examples, leukocyte migration in response to bacterial infection, particularly in a sepsis model, is reduced in the presence of PF4.

In some implementations, the subject is further administered an antibiotic compound, a non-antibiotic antibacterial substance, or a glucocorticoid. Because PF4 does not directly kill the bacteria, combination use with an agent that directly target bacteria like an antibiotic compound or a non-antibiotics antibacterial substance could result in additive if not synergistic bactericidal effects. In some aspects, the antibiotic compound administered is ceftriaxone, levofloxacin, or ampicillin.

Other features of PF4 make it a suitable molecule to target both antibiotic-susceptible and antibiotic-resistant pathogens include that PF4 exists in a monomer-dimer-tetramer equilibrium in solution. Therefore, due to its size (70 amino acids×4) and globular structure as compared to antibiotics and shorter antimicrobial peptides (linear; 5-40 amino acids) known in the prior art, PF4 only bind to but does not penetrate the capsule found in many types of bacteria, which decreases the possibility of the development of bacterial resistance to PF4. Also, unlike known antimicrobial peptides that often form the amphipathic helices which can insert into the bacterial and host cell membranes to result in cell lysis, PF4 is a globular protein that is incapable of lysing host cells. Thus, PF4 as an antimicrobial agent causes decreased, if not minimizes, the toxic effect on host cells associated with the use of known antimicrobial agent.

### Illustrative, Non-Limiting Examples in Accordance with Certain Embodiments

The disclosure is further illustrated by the following examples that should not be construed as limiting. The contents of all references, patents, and published patent applications cited throughout this application, as well as the Figures, are incorporated herein by reference in their entirety for all purposes.

### Example 1. PF4 Enhances Bacterial Phagocytosis In Vivo

A well-established model of bacterial peritonitis was used to determine whether PF4 can enhance bacterial phagocytosis by macrophages in vivo and thus reduce the number of viable bacteria. For the model of bacterial peritonitis, C57BL mice were inoculated intraperitoneally (i.p.) with S. aureus or with a mixture of S. aureus and PF4. Although PF4 is antimicrobial against both Gram-negative and Gram-positive bacteria, the selection of S. aureus for these studies was based on the fact that S. aureus is currently the predominant pathogen that causes the development of sepsis.

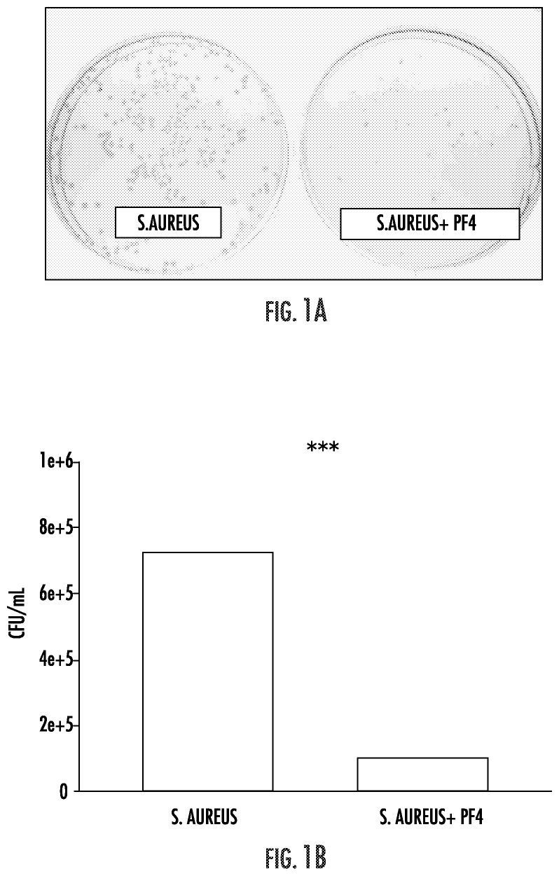

Specifically, mice were inoculated with S. aureus alone (1×10⁷ colony-forming units, CFU) or with a combination of endotoxin-free recombinant PF4 (20 μg) and S. aureus (1×10⁷ CFU). After 24 hours, mice were sacrificed, and peritoneal cavities were lavaged with 5 ml of a sterile endotoxin-free solution of PBS+5 mM EDTA. Samples of peritoneal lavage were serially diluted and plated on LB agar plates. After 16 hours of incubation at 37° C., colonies on the plates were counted (FIG. 1A). Data were expressed as CFU/ml recovered from peritoneal lavage. In the presence of PF4, the number of viable bacteria was decreased by 7.3±1.2 folds (FIGS. 1A and 1B).

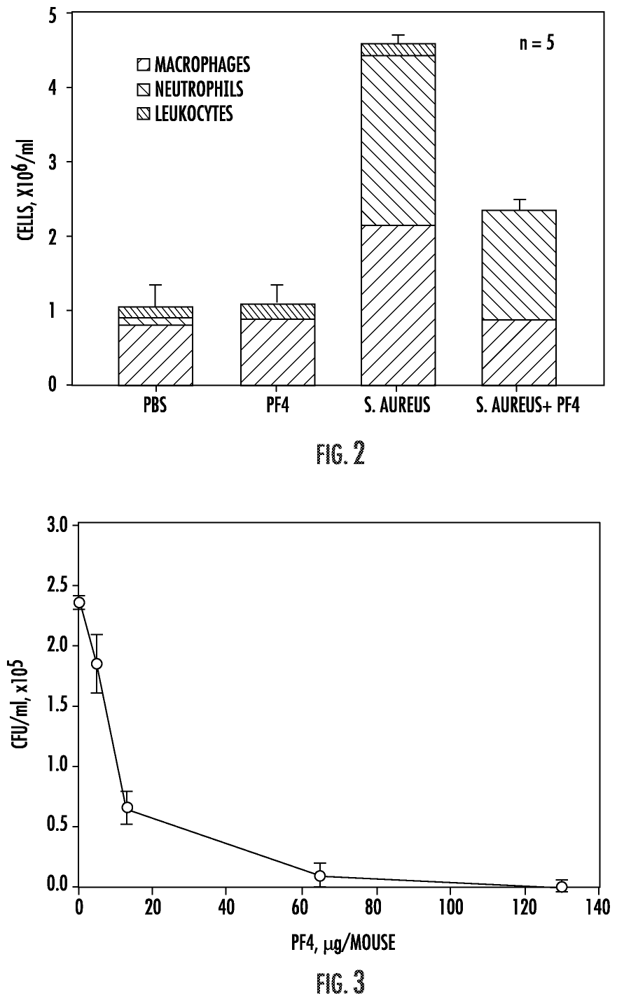

The retrieved peritoneum lavage was also analyzed for total leukocyte counts, and cytospin slides were used for differential counts of cells. The total number of leukocyte in the peritoneum was significantly elevated after S. aureus injection compared to control injection (4.6×10⁶/ml vs 1.1×10⁶/ml, respectively) (FIG. 2). The increase was primarily due to the neutrophil influx. Although the number of leukocytes was also increased after injection of S. aureus together with PF4, the increase was significantly less compared to injection of S. aureus alone (2.3×10⁶/ml vs 4.6×10⁶/ml, respectively) (FIG. 2). The injection of PF4 alone resulted in no increase in leukocyte numbers in comparison with control and no bacterial growth (FIG. 2).

### Example 2. PF4 Reduces the Number of Viable Bacteria in an Infected Subject in a Dose-Dependent Manner

C57BL mice were inoculated intraperitoneally with S. aureus alone (1×10⁷ CFU) or with varying concentrations of endotoxin-free recombinant PF4 (5-130 μg) and S. aureus (1×10⁷ CFU). After 24 hours, mice were sacrificed, and peritoneal cavities were lavaged with 5 ml of a sterile endotoxin-free solution of PBS+5 mM EDTA. Samples of peritoneal lavage were serially diluted and plated on LB agar plates. After 16 hours of incubation at 37° C., colonies on the plates were counted. Compared to the control, the addition of PF4 resulted in a marked decrease of the number of viable bacteria in a dose-dependent manner (FIG. 3, data were expressed as CFU/ml recovered from peritoneal lavage).

### Example 3. PF4 Increases the Survival Rate of Mice with Bacterial Peritonitis

C57BL mice were inoculated intraperitoneally with a sublethal dose of S. aureus alone (5×10⁸ CFU) or with a combination of endotoxin-free recombinant PF4 (13 μg) and S. aureus (5×10⁸ CFU). The mice were observed over a 9-day period. Analysis of the effect of PF4 on survival was performed using the Kaplan-Meier method (FIG. 4). The presence of PF4 decreases the mortality mice with bacterial peritonitis.

### Example 4. PF4 Reduces the Number of Viable Bacteria in a Subject Infected by Methicillin-Resistant S. aureus (MRSA)

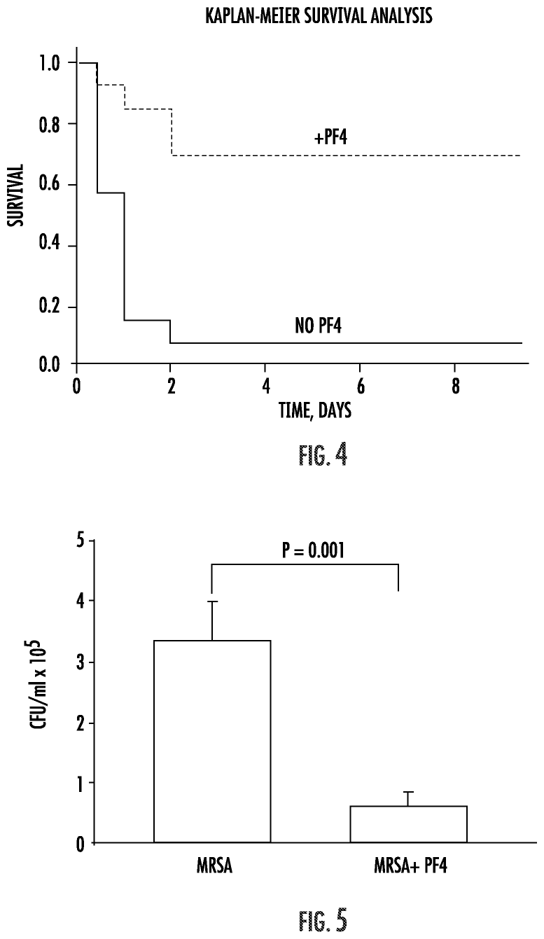

C57BL mice were inoculated intraperitoneally with MRSA alone (1×10⁷ CFU) or with a combination of endotoxin-free recombinant PF4 (13 μg) and MRSA (1×10⁷ CFU). After 24 hours, the mice were sacrificed, and peritoneal cavities were lavaged with 5 ml of a sterile endotoxin-free solution of PBS+5 mM EDTA. Samples of peritoneal lavage were serially diluted and plated on LB agar plates. After 16 hours of incubation at 37° C., colonies on the plates were counted. Data were expressed as CFU/ml recovered from peritoneal lavage (FIG. 5). In the presence of PF4, the number of viable bacteria was decreased by 5.7±1.4 folds (n=6).

1. A method of treating or inhibiting a bacterial infection in a subject in need thereof, the method comprising administering PF4 to the subject.

2. A method of reducing the severity of a bacterial infection in a subject in need thereof, the method comprising administering PF4 to the subject.

3. A method of inducing an immune response in response to a bacterial infection, the method comprising administering PF4 to a subject with the bacterial infection.

4. The method of claim 3, wherein the immune response induced is enhancing phagocytosis in response to the bacterial infection.

5. The method of claim 1, wherein the bacterial infection is caused by Gram-negative bacteria.

6. The method of claim 1, wherein the bacterial infection is caused by Gram-positive bacteria.

7. The method of claim 6, wherein the bacterial infection is caused by Staphylococcus aureus.

8. The method of claim 1, wherein the bacterial infection is caused by an antibiotic-resistant bacteria.

9. The method of claim 8, wherein the antibiotic-resistant bacteria is resistant to at least one antibiotic selected from the group consisting of: penicillin, oxacillin, methicillin, and amoxicillin-clavulanic.

10. The method of claim 8, wherein the antibiotic-resistant bacteria is methicillin-resistant S. aureus.

11. The method of claim 1, wherein the subject developed sepsis from the bacterial infection.

12. The method of claim 11, wherein the bacterial infection is caused by S. aureus.

13. The method of claim 1, wherein the subject developed peritonitis from the bacterial infection.

14. The method of claim 13, wherein the bacterial infection is caused by S. aureus.

15. The method of claim 1, further comprising administering to the subject an antibiotic compound, a non-antibiotic antibacterial substance, and/or a glucocorticoid.

16. The method of claim 3, wherein the bacterial infection is caused by Gram-negative bacteria.

17. The method of claim 3, wherein the bacterial infection is caused by Gram-positive bacteria.

18. The method of claim 17, wherein the bacterial infection is caused by Staphylococcus aureus.

19. The method of claim 3, wherein the bacterial infection is caused by an antibiotic-resistant bacteria.

20. The method of claim 3, further comprising administering to the subject an antibiotic compound, a non-antibiotic antibacterial substance, and/or a glucocorticoid.
